(19) United States
(12) Patent Application Publication
(10) Pub. No.: US 2010/0119408 A1
(43) Pub. Date: May 13, 2010

(54) ACTIVE DISPENSING SYSTEM AND METHOD

(75) Inventors: Kevin Hafer, Chandler, AZ (US); Jeffrey Bankers, Phoenix, AZ (US); Sanam Nassierpour, Phoenix, AZ (US)

Correspondence Address: SNELL & WILMER, LLP (Dial Corp), ONE ARIZONA CENTER, 400 E. VAN BUREN, PHOENIZ, AZ, 85004-2202, US

(73) Assignee: THE DIAL CORPORATION, Scottsdale, AZ (US)

(21) Appl. No.: 12/595,603

(22) PCT Filed: Apr. 11, 2008
(86) PCT No.: PCT/US08/59987

§ 371 Date: Oct. 12, 2009

(30) Foreign Application Priority Data
Apr. 13, 2007 (US) 60911768

(51) Int. Cl. A61L 9/00; F01N 3/28

(52) U.S. Cl. 422/4; 422/107

(57) ABSTRACT

The present invention provides a system for dispensing a volatizable material when needed. In an exemplary embodiment, the system includes a sensor to detect malodors, where the sensing of a malodor is used to activate fragrance emission when needed, and not, when not needed. In an exemplary embodiment, the sensor is able to distinguish between different malodors in different locations, such that a single system may be used in different locations. In an exemplary embodiment, the system includes an indicator configured to indicate to the sensor the type of malodor to detect to activate fragrance emission. In an exemplary embodiment, the indicator is included with the container of volatizable liquid, either mechanically or electrically.

### FIELD OF INVENTION

The present invention generally relates to vapor-dispensing devices. More particularly, the system may include active vapor-dispensing, based in part upon a sensor configured to detect different malodors. Furthermore, the system may include elements configured to communicate with other elements of the system, such as via a refill container which communicates to the system the particular malodor to detect.

### BACKGROUND OF THE INVENTION

Vapor-dispensing products typically include a volatizable material and a transport system configured to facilitate evaporation of the volatizable material into the surrounding air. For example, in some systems, a liquid material is contained in a reservoir bottle, and is volatized with heat, pump, aerosol, fan, etc.

Known vapor-dispensing devices of this type may be improved upon in a number of respects. For example, some vapor-dispensing devices dispense fragrance vapor at times when the fragrance may not be needed. Furthermore, some devices do not emit enough fragrance vapors when certain malodors are present. Additionally, certain malodors associated with different locations, i.e., kitchen, bathroom, laundry room, locker, children's closet, etc., may be treated more effectively with certain fragrances.

### SUMMARY OF THE INVENTION

The present invention provides a system for dispensing a volatizable liquid when needed. In an exemplary embodiment, the system includes a sensor to detect malodors, where the sensing of a malodor is used to activate fragrance emission when needed, and not, or less so, when not needed. In an exemplary embodiment, the sensor is able to distinguish between different malodors in different locations, such that a single system may be used in different locations. In an exemplary embodiment, the system includes an indicator configured to indicate to the sensor the type of malodor to detect to activate fragrance emission. In an exemplary embodiment, the indicator may be included with the container of volatizable liquid, either mechanically or electrically.

### BRIEF DESCRIPTION OF THE DRAWINGS

These and other features, aspects and advantages of the present invention will become better understood with reference to the following description, appending claims, and accompanying drawings where:

FIG. 1 is a block diagram of a system for active dispensing, according to an exemplary embodiment;

FIG. 2 is a side elevational view of a replacement cartridge, according to an exemplary embodiment;

FIG. 3 is a block diagram of a system for active dispensing, according to an exemplary embodiment;

FIG. 4 is a block diagram and flow diagram of a system and method of active dispensing, according to an exemplary embodiment;

FIG. 5 is a flow diagram of a method of active dispensing, according to an exemplary embodiment.

### DETAILED DESCRIPTION

The following descriptions are of exemplary embodiments of the invention only, and are not intended to limit the scope or applicability of the invention in any way. Rather, the following description is intended to provide convenient illustrations for implementing various embodiments of the invention. As will become apparent, various changes may be made without departing from the spirit or scope of the invention as set forth in the appended claims.

For the sake of brevity, functional embodiments of the apparatus and systems (and components of the individual operating components of the systems) may not be described in detail herein. Furthermore, the connecting lines shown in the various figures contained herein are intended to represent exemplary functional relationships or physical connections between the various elements. It should be noted that many alternative or additional functional relationships or physical connections may be present in a practical system.

Exemplary embodiments of the present invention include an active dispensing system in which elements of the system communicate with other elements of the system. The information may include attributes about the elements themselves, information about the operating mode of the system, information about the operation of the system or other information. In accordance with various embodiments, the communication allows the system to operate more effectively.

The communication may be mechanical or non-mechanical. In accordance with embodiments comprising mechanical communication between elements, the mechanical indicators can include collars, molding protrusions, keyed fitments, other mechanical configurations or combinations thereof. In various embodiments, the mechanical indicators are configured to actuate a switch that initiates a change in the utility or method of operation of the system.

In an exemplary embodiment, a mechanical indicator is in the form of protrusions (“keys”) on a refill cartridge. In accordance with this particular embodiment, the protrusions are configured to actuate actuators within the system, for example, in a keyed-type relationship. In various embodiments, the information communicated includes attributes about the volatizable material within the refill cartridge. In various embodiments, the information communicated includes attributes about the location the system is being used in, what malodor to detect, and/or what volatizable material to detect.

In accordance with embodiments comprising non-mechanical communication between elements, the non-mechanical indicators can comprise electrical, wireless (e.g., RFID), other non-mechanical configurations or combinations thereof. Exemplary non-mechanical indicators are positioned within the system so as to close a communication circuit and thereby initiate a change in the utility or method of operation of the system.

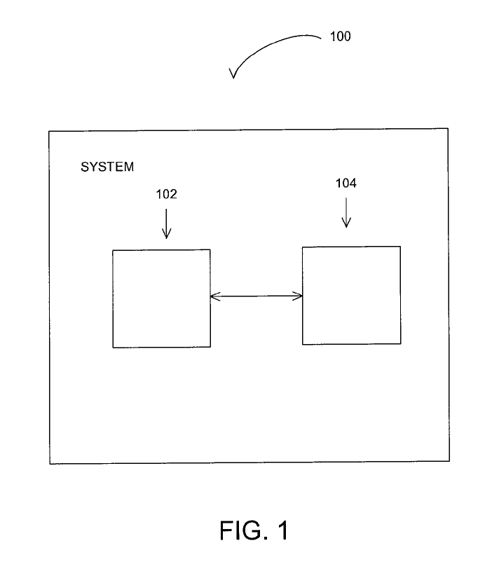

That being said, FIG. 1 is a block diagram of an active dispensing system 100, according to an exemplary embodiment. In this embodiment, system 100 includes a first element 102 and a second element 104.

In this embodiment, elements 102 and 104 are configured to communicate information. The information may include attributes about the elements themselves, information about the operating mode of the system, information about the operation of the system or other information. Elements 102 and 104 may be any of the elements of the system.

In an exemplary embodiment, one such element is a refill cartridge or container. In accordance with various aspects of an exemplary embodiment, the refill container communicates to the system the type of volatizable material within the refill container. This information is used by the system to determine the particular type of malodor to detect. If the type of particular type of malodor is detected, the system then dispenses the volatizable material to treat the area.

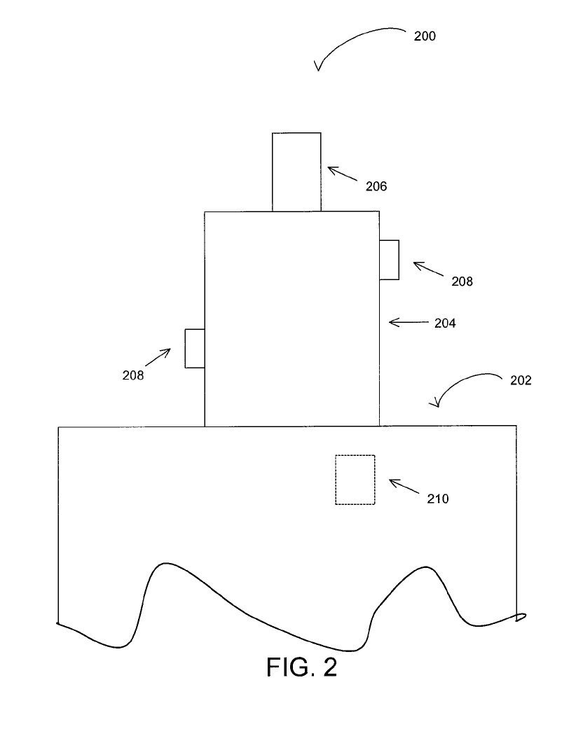

FIG. 2 is a side elevational view of a replacement cartridge 200, according to an exemplary embodiment of an active dispensing system. Cartridge 200 may include a container 202, a cap 204, a wick 206, mechanical indicators 208 or a non-mechanical indicator 210. In exemplary embodiments, the indicator, as discussed below, is configured to indicate to the system which malodors to detect and, upon detection, activate dispensing system.

In an exemplary embodiment, container 202 is configured to contain a volatizable material, such as a liquid (e.g., oil), solid (e.g., gel) or other suitable material. In an exemplary embodiment, cap 204 is configured to seal or cap container 202, and support or contain wick 206.

In an exemplary embodiment, wick 206 extends through cap 204 into container 202, such that it is in contact with the volatizable material therein. In this manner, the volatizable material saturates or semi-saturates wick 206 to allow the volatizable material to be exposed to a dispensing system to enhance the dispensing of the volatizable material or fragrance.

In one embodiment, cap 204 includes mechanical indicators 208. Mechanical indicators 208 may be configured to indicate to a sensor or the system which malodor to detect to activate the dispensing system. Mechanical indicators 208 may also actuate switches to indicate to a sensor or the system which malodor to detect to activate the dispensing system. In this manner, the system may be configured to detect and treat specific malodors common in certain areas, for example, a home, office, locker room, etc.

In another embodiment, cartridge 200 includes a non-mechanical indicator 210. Non-mechanical indicator 210 may be configured to indicate to a sensor or system which malodor to detect to activate the dispensing system. In accordance with an aspect of an exemplary embodiment, electrical communication includes circuitry or other electric components in cartridge 200 which, when in communication with the system, provide an indication of the volatizable material within the cartridge 200, the location of the system, what malodor to detect, what volatizable material to detect, or the utility or method of operation of the system.

In general, the forms of communication between cartridge 200 and the system comprise, but are not limited to mechanical and non-mechanical (e.g., electrical).

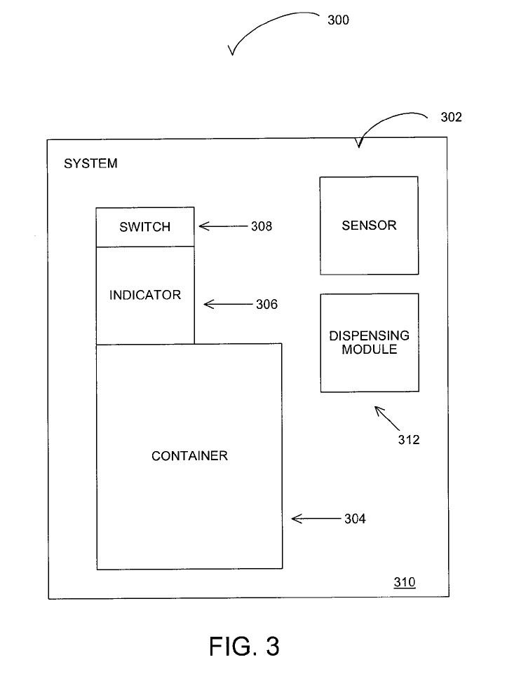

FIG. 3 is a block diagram of an active dispensing system 300, according to an exemplary embodiment. In this embodiment, system 300 includes a sensor 302, a container 304, an indicator 306, and a switch 308. In various embodiments, system 300 also comprises a dispensing module 312 and/or a housing 310, which may enclose, either partially or entirely, the other elements of the system.

In an exemplary embodiment, sensor 302 is configured to detect the presence or intensity of a plurality of malodors, and, optionally, the presence or intensity of materials dispensed by system 300, such as various volatizable materials or fragrances. As discussed above, different malodors may be present in different areas, for example, a home, office, locker room, etc. Sensor 302 can be of many different types, capable of sensing detecting one or a plurality of malodors. In an exemplary embodiment, sensor 302 is a metal oxide sensor (MOS), however, it should be understood by one of ordinary skill in the art that any suitable sensor exhibiting the qualities discussed herein may be used in the context of the present invention.

In an exemplary embodiment, system 300 includes a container 304, which may be configured to contain a volatizable material, such as a liquid (e.g., aromatic oil), solid (e.g., gel) or other suitable material.

In an exemplary embodiment, system 300 includes an indicator 306. In one particular embodiment, indicator 306 is a mechanical indicator, such as bumps located on container 304 that depress actuators within housing 310. However, indicator 306 may be mechanical or non-mechanical. In accordance with an aspect of various embodiments, indicator 306 indicates to sensor 302 what malodor to detect to activate fragrance emission. If sensor 302 detects an objectionable malodor, then sensor 302 sends an indication to dispensing module 312 to dispense the volatizable material or fragrance. In accordance with other aspects of various embodiments, indicator 306 conveys attributes about the volatizable material within container 304 or what volatizable material to detect.

In an exemplary embodiment, system 300 includes a switch 308. In one particular embodiment, switch 308 is multi-positioned, such that one position may be for the kitchen, while others may be for the bathroom, laundry room, etc. In various embodiments, switch 308 indicates to system 300 the volatizable material within container 304, the location of system 300, what malodor to detect, what volatizable material to detect, or the utility or method of operation of the system.

In one embodiment, system 300 operates in a mode in which sensor 302 gives an indication to dispensing module 312 to dispense fragrance. In another embodiment, switch 308 is connected to a timer to indicate to dispensing module 312 to dispense fragrance at pre-set intervals. Furthermore, switch 308 may indicate an immediate activation of dispensing module 312 to mask, neutralize, or otherwise remove a malodor. For example, in accordance with various exemplary embodiments, switch 308 is a button, switch or other actuator that acts to initiate an immediate burst of volatizable material or fragrance.

In an exemplary embodiment, system 300 and a related method include a sensor or plurality of sensors configured to distinguish between a first malodor and a second malodor. The system and method may include a first reservoir configured for storage of a first air treatment, and a second reservoir configured for storage of a second air treatment, different from the first. In accordance with one aspect of an exemplary embodiment, a delivery system may be configured to dispense the first air treatment in response to the sensor identifying the first malodor and dispense the second air treatment in response to the sensor identifying the second malodor.

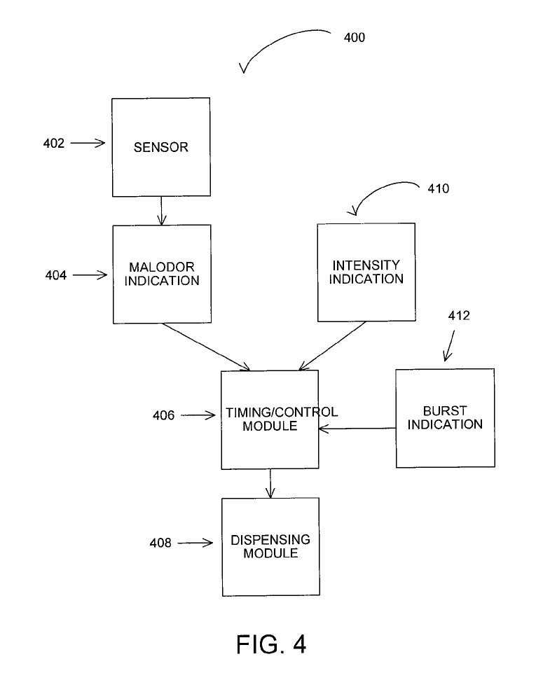

FIG. 4 is a flow diagram of a method and system 400 of active dispensing of an air treatment, according to an exemplary embodiment. In various exemplary embodiments, method and system 400 dispense an air treatment in many different manners, such as the three shown in FIG. 4. In accordance with one exemplary embodiment, system 400 dispenses an air treatment includes a sensor 402 sensing an activation malodor and sending a malodor indication 404 to a timing/control module 406. Any suitable sensor, including those discussed above, exhibiting the qualities discussed herein may be used. The timing/control module 406 would then send a dispensing signal to a dispensing module 408 to indicate to the dispensing module 408 to dispense a volatizable liquid or fragrance. This is one mode of operation for system 400.

In accordance with another exemplary embodiment, system 400 includes the setting of an intensity indication 410. The intensity indication 410 may be set by a user, which would send an activation signal to the timing/control module 406 to periodically activate the dispensing module 408 and optionally, at a selected intensity. In an exemplary embodiment, the timing/control module 406 may contain interval timed programming wherein air treatment material is dispersed in predetermined intervals during the timed treatment. This may be a user preference, in that a volatizable liquid or fragrance would be dispensed at regular periodic intervals and at a desired intensity, both of which may be increased or decreased to certain limits, depending on the consumer's desired treatment intervals and intensity. In accordance with one aspect of an exemplary embodiment, this is a factory setting, pre-set before the system is activated.

In accordance with yet another exemplary embodiment, system 400 includes a burst indication 412. The burst indication 412 may be initiated by an actuator on the system to immediately dispense the air treatment. The burst indication 412 would communicate with the timing/control module 406, which would then activate the dispensing module 408 to relatively immediately dispense the air treatment. In a particular embodiment, a user actuates a button or another indication is provided, which signals that immediate dispensing of an air treatment is desired. This may further provide an air treatment to mask, neutralize or otherwise remove malodors within the area.

In addition to dispensing an air treatment and in accordance with various embodiments, dispensing module 408 also comprises an aerosol or other method of rapid deploying an air treatment within the area. For example, system 400 may include a multi-position switch to indicate to the system which mode to operate in. Importantly, each mode may function alone or in combination with one or more of a sensor mode, an intensity indication mode and a burst indication mode.

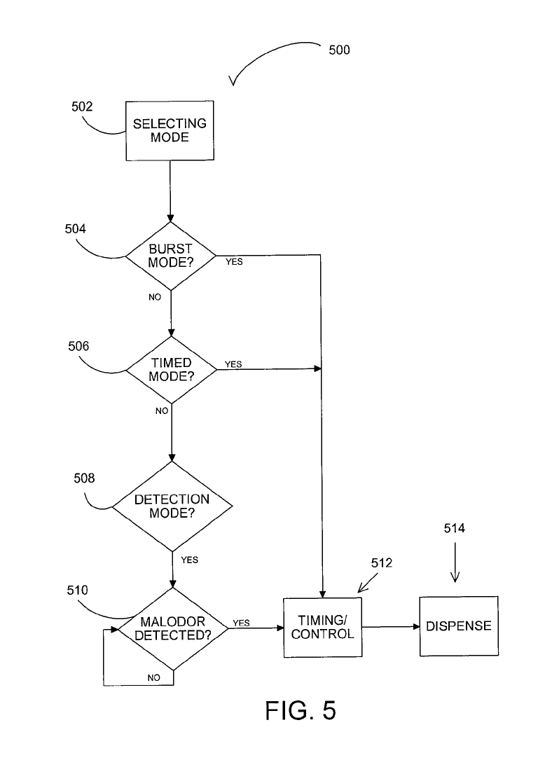

FIG. 5 shows a flow diagram of a method 500 of active dispensing, according to an exemplary embodiment. In this embodiment, method 500 includes selecting a mode 502, determining if it is a burst mode 504, determining if it is a timed mode 506, determining if it is a detection mode 508, malodor detection 510, performing timing and control 512, and dispensing 514.

Selecting a mode 502, in an embodiment, includes selecting a mode of operation of an air treatment device. This may be done manually by a user, or may be generally automatic. The device may comprise any number of modes, from one to hundreds or more. Each mode may function alone or in combination with another or others. In this embodiment there are three modes.

After selecting a mode 502, a determination is made of whether the system is in a burst mode 504. If the system is in a burst mode 504, the YES leg is taken to the timing and control step 512 followed by the dispensing step 514, which would, relatively immediately, dispense an air treatment in a burst or aerosol configuration. In an exemplary embodiment, in a burst mode 504, the consumer may activate the burst mode 504, relatively independent of a timed mode 506 or a detection mode 508. Selecting burst mode 504 may or may not reset the timed mode 506 or deactivate the detection mode 508 for a time period, depending on the air treatment and its method of delivery. If the burst mode determination is negative, the NO leg is taken to determine whether the system is in a timed mode 506.

If the system is in a timed mode 506, the YES leg is taken to the timing and control step 512 followed by the dispensing step 514. In an exemplary embodiment of a system in a timed mode 506, the timing and control step 512 periodically sends a signal to the dispensing step 514. This periodic signaling may be preset or may be customizable by the user in terms of duration as well as intensity. If the timed mode determination is negative, the NO leg is taken to determine whether the system is in a detection mode 508.

If the system is in a detection mode 508, the YES leg is taken to the malodor detection step 510. If a particular type of malodor is detected 510, the YES leg is taken to the timing and control step 512 followed by the dispensing step 514. If the system is in a detection mode 508 and a malodor is not detected 510, the NO leg is taken and the malodor detection step 510 continues until a malodor is detected 510, upon which the YES leg is taken to the timing and control step 512 followed by the dispensing step 514.

In an exemplary embodiment, upon activation (or at any point during operation) the consumer sets the mode in which the air treatment system will operate via the selection mechanism. Next, the timing control circuit activates the air treatment device according to the selection mechanism. The selection mechanism controls the duration of the treatment, “on time” and the intervening time between treatments, “off time” thereby allowing the delivery system to dispense air treatment according to the desired level chosen by the consumer.

Additionally, during operation of a sensing treatment, the sensor and its control circuitry may also continuously sample the environment for either the level of active air treatment material or the environmental air quality. The level of active air treatment material is sampled by the sensor so as to maintain the desired level of environmental air quality. The environmental air quality is sampled by the sensor so that in response to a decrease in the desired environmental air quality, the quantity of air treatment material distributed may be increased. If the predetermined level of environmental air quality is not satisfactory, i.e. the sensor detects a malodor, the sensor communicates with the timing control circuit to provide an increase in air treatment. Specifically, the sensor detects a malodor and sends an indicator signal to the timing control circuit. The timing control circuit then actuates the delivery system accordingly. This action continues until the sensor determines the environmental air quality has returned to the desired level of environmental air quality or the timing control circuit has determined that a significant enough period of increased air treatment has occurred. The sensor determines the desired level of environmental air quality by monitoring the air quality and sending indicator signals to the timing control circuit until the desired level is reached.

During operation of a timed treatment, the duration and intervening time are set according to the selection mechanism and dispense air treatment accordingly. It should be understood that the air treatment system dispenses air treatment according to timed intervals or in response to detection of a malodor.

Further, if the timing control circuit determines a significant period of increased air treatment, the timing control circuit disengages the sensor for an operational time period via the feedback loop. After the time period, the sensor re-engages and once again begins sampling. Additionally, the sensor communicates with the timing control circuit through the feedback loop to determine when the air treatment delivery system has been actuated and, thus, allows the sensor sufficient time to be either off (if the consumer is sensitive to initiating the air treatment system) or to correct the baseline of the sensor response to account for the air treatment system.

It should be understood that the foregoing description is of exemplary embodiments of the invention only, and that the invention is not limited to the specific forms shown. Various modifications may be made in the design and arrangement of the elements set forth herein without departing from the scope of the invention.

What is claimed is:

1. An air treatment device for dispensing a volatizable material comprising:
a housing;
a sensor configured to distinguish between a first malodor and a second malodor;
a timing control circuit coupled with said sensor;
a delivery system coupled with said timing control circuit; and
a refill configured for storage of a volatizable material, wherein said refill is in communication with said sensor and is configured to indicate a volatizable material to said sensor and wherein said sensor is configured to associate said first malodor with said volatizable material.

2. A device as in claim 1, wherein said refill is mechanically keyed to indicate said volatizable material to said sensor.

3. An active dispensing system comprising:
a housing;
a sensor configured to distinguish between a first malodor and a second malodor;
a timing control circuit coupled with said sensor;
a delivery system coupled with said timing control circuit;
a first reservoir configured for storage of a first air treatment; and
a second reservoir configured for storage of a second air treatment;
wherein said delivery system is configured to dispense said first air treatment in response to said sensor identifying said first malodor and dispense said second air treatment in response to said sensor identifying said second malodor.

4. A device as in claim 3 wherein said sensor is a metal oxide sensor.

5. A method for dispensing an appropriate volatizable material when needed comprising:
a refill indicating a volatizable material to a sensor, wherein said sensor is configured to distinguish between a first malodor and a second malodor;
said sensor associating said first malodor with said volatizable material;
said sensor detecting said first malodor;
said sensor communicating with a timing control circuit;
said timing control circuit communicating with a delivery system; and
said delivery system dispensing said volatizable material.

6. A method as in claim 5, wherein said step of said timing control circuit communicating with a delivery system further comprises said timing control circuit disengaging said sensor for an operational time period via a feedback circuit.

7. A method as in claim 6, wherein said step of said timing control circuit disengaging said sensor for an operational time period via a feedback circuit further comprises said timing control circuit disengaging said sensor if said timing control circuit determines a significant time period of increased air treatment.

8. A method as in claim 5, wherein said step of said timing control circuit communicating with a delivery system further comprises said timing control circuit providing said delivery system with interval timed programming wherein said air treatment is dispensed in predetermined intervals.

9. A method for dispensing different air treatments when needed comprising:
detecting a first malodor with a sensor, wherein said sensor is configured to distinguish between said first malodor and a second malodor;
detecting a second malodor with said sensor;
said sensor communicating with a timing control circuit;
said timing control circuit communicating with a delivery system; and
said delivery system dispensing a first air treatment in response to said sensor identifying said first malodor and dispensing a second air treatment in response to said sensor identifying said second malodor.
